(19) United States
(12) Patent Application Publication
(10) Pub. No.: US 2023/0242873 A1
(43) Pub. Date: Aug. 3, 2023

(54) FIBROBLAST BASED THERAPY FOR TREATMENT OF PARKINSON'S DISEASE

(71) Applicant: FIGENE, LLC, Houston, TX (US)

(72) Inventors: Pete O'HEERON, Houston, TX (US); Thomas ICHIM, San Diego, CA (US)

(21) Appl. No.: 17/998,183

(22) PCT Filed: May 13, 2021
(86) PCT No.: PCT/US2021/070551

§ 371 Date: Nov. 8, 2022

Related U.S. Application Data
(60) Provisional application No. 63/024,440, filed on May 13, 2020.

(51) Int. Cl. C12N 5/0793; A61K 35/33

(52) U.S. Cl. CPC C12N 5/0619; A61K 35/33; C12N 2506/1307; C12N 2501/999; C12N 2501/13

(57) ABSTRACT

In some aspects, disclosed herein are methods and compositions for treatment of Parkinson's disease using fibroblasts or cells derived from fibroblasts. Also disclosed herein are methods and compositions for generating dopaminergic cells from fibroblasts. Dopaminergic cells generated from fibroblasts are described. Methods of the present disclosure include methods for treatment or preventing of Parkinson's disease comprising the use of fibroblasts or dopaminergic cells generated from fibroblasts.

This application claims priority to U.S. Provisional Patent Application Ser. No. 63/024,440, filed May 13, 2020, which is incorporated by reference herein in its entirety.

### TECHNICAL FIELD

Embodiments of the disclosure generally include at least the fields of cell biology, neurobiology, and medicine. More particularly, the disclosure pertains to the area of cellular differentiation and the generation of neuronal cells for treatment of Parkinson's Disease, specifically, the generation of dopaminergic cells from fibroblasts.

### BACKGROUND

Parkinson's disease (PD) is a degenerative disorder of the central nervous system, which results from the death of dopamine-generating (DA) neurons in the substantia nigra, a region of the midbrain. PD affects nearly a million Americans, with 50,000 new cases diagnosed in the U.S. each year. PD is the second most common neurodegenerative disorder after Alzheimer's disease. The prevalence of PD is about 0.3% of the whole population in industrialized countries. While genetically linked familial PD has early onset (between the ages of 20 and 50), the more common sporadic PD occurs after the age of 50 (average 57.+−0.11). PD is more common in the elderly and prevalence rises from 1% in those over 60 years of age to 4% of the population over 80. The incidence of PD has been estimated between 8 and 18 per 100,000 person years. The main motor symptoms of PD are collectively called parkinsonism, or a “parkinsonian syndrome”. Early in the course of the disease, the most obvious symptoms are movement-related, such as shaking, rigidity, postural instability, slowness of movement, difficulty with walking, impaired gait, and tendency to fall. Later, cognitive and behavioral problems may arise, with dementia commonly occurring in the advanced stages of the disease. Other symptoms include sensory, sleep and emotional problems. Generally, accompanying the other characteristics of the fully developed disorder is the festinating gait, whereby the patient, prevented by the abnormality of postural tone from making the appropriate reflex adjustments required for effective walking, progresses with quick shuffling steps at an accelerating pace.

Although modern treatments of Parkinson's disease exist, such as levodopa and stereotactic surgery, there are still many challenges in effectively treating this disease. Underlying much of the difficulty is the fact that none of these therapeutic measures has an effect on the underlying disease process, which consists of neuronal degeneration.

The present disclosure satisfies a long felt need in the art for methods and compositions for the treatment of neuronal degeneration, such as degeneration underlying Parkinson's disease, as well as improved methods and compositions for treatment of Parkinson's disease symptoms.

### BRIEF SUMMARY

The present disclosure, in some embodiments, is directed to methods and compositions related to treating or preventing Parkinson's disease. Disclosed herein are methods and compositions for generation of dopaminergic cells from one or more fibroblasts. In particular embodiments, compositions of the present disclosure comprise dopaminergic cells generated from culturing fibroblasts under appropriate conditions. Dopaminergic cells may be used to treat or prevent Parkinson's disease in an individual. In some embodiments, fibroblasts are used to treat or prevent Parkinson's disease in an individual.

Embodiments include dopaminergic cells generated from fibroblasts, fibroblasts capable of treating Parkinson's disease, and compositions capable of inducing transdifferentiation of fibroblasts to dopaminergic cells. Embodiments include methods for treating Parkinson's disease, methods for preventing Parkinson's disease, methods for increasing a number of dopaminergic cells in an individual, methods for improving motor function, methods for generating dopaminergic cells, methods for differentiating fibroblasts into neuronal cells, methods for transdifferentiation of fibroblasts, and methods for providing dopaminergic cells to an individual.

Methods of the present disclosure can include at least 1, 2, 3, 4, 5, 6, or more of the following steps: subjecting fibroblasts to conditions sufficient to generate dopaminergic cells from the fibroblasts, providing fibroblasts to an individual, providing dopaminergic cells to an individual, culturing fibroblasts in all-trans retinoic acid, culturing fibroblasts with a neurotrophic factor, isolating fibroblasts from an individual, and purifying neuronal cells from a cell culture. Compositions of the present disclosure can include one or more of dopaminergic cells, neuronal cells, fibroblasts, and derivatives thereof.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this

### DETAILED DESCRIPTION

In some embodiments, provided herein is a method for treating or preventing Parkinson's disease in an individual comprising administering an effective amount of a composition comprising dopaminergic fibroblast cells to an individual in need thereof. In some embodiments, provided herein is a method for generating dopaminergic cells from fibroblasts comprising subjecting fibroblasts to conditions sufficient to generate dopaminergic cells from the fibroblasts.

In some embodiments, the fibroblast cells are cultured under conditions sufficient to differentiate the fibroblasts into neuronal cells. In some embodiments, the conditions comprise treatment of the fibroblasts with one or more agents capable of activating tyrosine hydroxylase expression in the fibroblasts. In some embodiments, the conditions comprise culturing the fibroblasts with all-trans retinoic acid (RA). In some embodiments, the conditions further comprise culturing the fibroblasts with one or more growth factors. In some embodiments, the growth factor is one or more neurotrophic factors. In some embodiments, the one or more neurotrophic factors comprise brain-derived neurotrophic factor (BDNF), glial cell line-derived neurotrophic factor (GDNF), nerve growth factor (NGF), a fibroblast growth factor (FGF), or a combination thereof.

In some embodiments, the dopaminergic cells express tyrosine hydroxylase. In some embodiments, the dopaminergic cells express the tyrosine hydroxylase transiently. In some embodiments, the dopaminergic cells express NeuN. In some embodiments, the dopaminergic cells do not express CD40. In some embodiments, the dopaminergic cells express Nestin.

In some embodiments, the Parkinson's disease comprises immunologically mediated loss of endogenous dopaminergic cells in the individual. In some embodiments, the Parkinson's disease comprises non-immunologically mediated loss of endogenous dopaminergic cells in the individual.

In some embodiments, the dopaminergic cells are provided to the individual intracranially. In some embodiments, the dopaminergic cells are provided to the putamen of the individual.

In some embodiments, the individual has been diagnosed with Parkinson's disease. In some embodiments, the individual is at risk for developing Parkinson's disease. In some embodiments, the individual at risk of developing Parkinson's disease includes male individuals, individuals age 60 and older, individuals with a genetic predisposition, individuals who have been exposed to environmental toxins, and/or individuals with a history of head trauma. In some embodiments, the Parkinson's disease comprises corticobasal degeneration, dementia with Lewy bodies, drug-induced parkinsonism, essential tremor, multiple system atrophy, progressive supranuclear palsy, vascular parkinsonism, or a combination thereof. In some embodiments, the fibroblasts are exposed to inflammatory conditions, and wherein the fibroblasts reduce inflammation in the individual.

In some embodiments, the fibroblasts are fibroblasts isolated from placenta, cord blood, peripheral blood, omentum, hair follicle, skin, bone marrow, adipose tissue, or Wharton's Jelly. In some embodiments, the fibroblasts are fibroblasts isolated from peripheral blood of a subject who has been exposed to conditions sufficient to stimulate fibroblasts from the subject to enter the peripheral blood. In some embodiments, the conditions sufficient to stimulate fibroblasts from the subject to enter the peripheral blood comprise administration of VLA-5 antibodies, G-CSF, M-CSF, GM-CSF, FLT-3L, TNF-α, EGF, FGF-1, FGF-2, FGF-5, VEGF, or a combination thereof.

In some embodiments, provided herein is a dopaminergic cell, wherein the dopaminergic cell is derived from a fibroblast that was cultured with all-trans retinoic acid (RA) and one or more neurotrophic factors. In some embodiments, the dopaminergic cell expresses tyrosine hydroxylase. In some embodiments, the dopaminergic cell expresses the tyrosine hydroxylase transiently. In some embodiments, the dopaminergic cell expresses NeuN. In some embodiments, the dopaminergic cell does not express CD40. In some embodiments, the dopaminergic cell expresses Nestin. In some embodiments, the one or more neurotrophic factors comprise brain-derived neurotrophic factor (BDNF), glial cell line-derived neurotrophic factor (GDNF), nerve growth factor (NGF), a fibroblast growth factor (FGF), or a combination thereof.

In some embodiments, the fibroblast from which the dopaminergic cell was derived as isolated from placenta, cord blood, peripheral blood, omentum, hair follicle, skin, bone marrow, adipose tissue, or Wharton's Jelly. In some embodiments, the fibroblast was isolated from peripheral blood of a subject who has been exposed to conditions sufficient to stimulate fibroblasts from the subject to enter the peripheral blood. In some embodiments, the conditions sufficient to stimulate fibroblasts from the subject to enter the peripheral blood comprise administration of VLA-5 antibodies, G-CSF, M-CSF, GM-CSF, FLT-3L, TNF-α, EGF, FGF-1, FGF-2, FGF-5, VEGF, or a combination thereof.

Other embodiments of the disclosure are discussed throughout this application. Any embodiment discussed with respect to one aspect of the disclosure applies to other aspects of the disclosure as well and vice versa. Each embodiment described herein is understood to be embodiments of the disclosure that are applicable to all aspects of the disclosure. It is contemplated that any embodiment discussed herein can be implemented with respect to any method or composition of the disclosure, and vice versa. Furthermore, compositions and kits of the disclosure can be used to achieve methods of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

As used herein, “cell culture” means conditions wherein cells are obtained (e.g., from an organism) and grown under controlled conditions (“cultured” or grown “in culture”) outside of an organism. A primary cell culture is a culture of cells taken directly from an organism (e.g., tissue cells, blood cells, cancer cells, neuronal cells, fibroblasts, etc.). Cells are expanded in culture when placed in a growth medium under conditions that facilitate cell growth and/or division. Various growth media may be used for the purposes of the present disclosure including, for example, Dulbecco's Modified Eagle Media (also known as Dulbecco's Minimal Essential Media) (DMEM), or DMEM-low glucose (also DMEM-LG herein). DMEM-low glucose may be supplemented with fetal bovine serum (e.g., about 10% v/v, about 15% v/v, about 20% v/v, etc), antibiotics, antimycotics (e.g., penicillin, streptomycin, and/or amphotericin B), and/or 2-mercaptoethanol. Cells may be cultured at 37 ° C. in a standard humidified atmosphere comprising 5% CO₂. While such conditions are useful for culturing, it is to be understood that such conditions are capable of being varied by the skilled artisan who will appreciate the options available in the art for culturing cells. When cells are expanded in culture, the rate of cell proliferation is sometimes measured by the amount of time needed for the cells to double in number. This is referred to as doubling time.

“Differentiation” (e.g., cell differentiation) describes a process by which an unspecialized (or “uncommitted”) or less specialized cell acquires the features (e.g., gene expression, cell morphology, etc.) of a specialized cell, such as a nerve cell or a muscle cell for example. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. In some embodiments of the disclosure, “differentiation” of fibroblasts to neuronal cells is described. This process may also be referred to as “transdifferentiation”.

As used herein, “dedifferentiation” refers to the process by which a cell reverts to a less specialized (or less committed) position within the lineage of a cell. As used herein, the lineage of a cell defines the heredity of the cell. The lineage of a cell places the cell within a hereditary scheme of development and differentiation. Within the context of the current disclosure, “dedifferentiation” may refer to fibroblasts acquiring more “immature” associated markers such as OCT4, NANOG, and SOX2. Additionally, “dedifferentiation” may mean acquisition of functional properties such as enhanced proliferation activity and/or migration activity towards a chemotactic gradient. In some embodiments fibroblasts may be “dedifferentiated” by treatment with various conditions, subsequent to which they are “differentiated” into other cell types, for example neuronal cells such as dopamine producing cells.

“Dopaminergic cells” describe cells which possess a dopaminergic phenotype. A dopaminergic phenotype includes expression of one or more of tyrosine hydroxylase, aldehyde dehydrogenase 2 (ALDH2), DARPP-32, and D2 dopamine receptor.

“Fibroblasts” refer to a cell, progenitor cell, or differentiated cell and include isolated fibroblast cells or population(s) thereof capable of proliferating and differentiating into ectoderm, mesoderm, or endoderm. In some embodiments, fibroblasts are utilized in an autologous, allogenic, or xenogenic manner. As used herein, placental and adult-derived cellular populations are included in the definition of “fibroblasts.” In the context of the present disclosure, fibroblast cells may be derived through means known in the art from sources including at least foreskin, ear lobe skin, bone marrow, cord blood, placenta, amnion, amniotic fluid, umbilical cord, embryos, intraventricular cells from the cerebral spinal fluid, circulating fibroblast cells, mesenchymal stem cell associated cells, germinal cells, adipose tissue, exfoliated tooth-derived fibroblasts, hair follicle, dermis, skin biopsy, nail matrix, parthenogenically-derived fibroblasts, fibroblasts that have been reprogrammed to a dedifferentiated state, side population-derived fibroblasts, fibroblasts from plastic surgery-related by-products, and the like.

“Parkinson's disease” (PD) and parkinsonism refer collectively to neurodegenerative syndromes in which parkinsonism related movement disorders may be a feature. These disorders, both genetic and non-genetic, may be characterized by four primary parkinsonian symptoms: tremor, rigidity, postural instability and bradykinesia, the symptoms resulting from the loss or dysfunction of dopamine-producing neurons in the substantia nigra. Parkinsonism may result from idiopathic Parkinson's disease, or may be caused in whole or in part by other factors, including but not limited to, medication, Alzheimer's disease, progressive supranuclear palsy, multiple system atrophy, Shy-Drager syndrome, olivo-ponto-cerebellar atrophy, general striatonigral degeneration or, less commonly, hydrocephalus, Wilson's disease, cortico-basal degeneration, Huntington's disease, Hallervorden-Spatz disease, post-encephalitic parkinsonism, manganese poisoning, pesticide exposure, and carbon monoxide poisoning.

Individuals at risk of developing Parkinson's disease include male individuals, individuals age 60 and older, individuals with a genetic predisposition, individuals who have been exposed to environmental toxins, and/or individuals with a history of head trauma. Age is a primary risk factor for developing Parkinson's disease, with an average age of onset of 60. Men are more likely to develop Parkinson's disease than women, and individuals with a parent or sibling who is affected with Parkinson's disease have roughly twice the chance of developing Parkinson's disease compared to individuals without affected parents or siblings. Genes known to contribute to Parkinson's disease include SNCA, PARK2, PARK7, PINK1, and LRRK2, though several as-yet discovered genes are also likely to play a role. Studies have also suggested a link between exposure to certain environmental toxins like pesticides and/or herbicides, MPTP, Agent Orange, manganese and other metals, solvents, and organic pollutants and an increased risk of developing Parkinson's disease. Repeated blows to the head can also increase an individual's risk of developing Parkinson's disease.

The term parkinsonism is also intended to include related disorders such as essential tremor and vascular pseudo-parkinsonism. In some embodiments, Parkinson's disease comprises corticobasal degeneration, dementia with Lewy bodies, drug-induced parkinsonism, essential tremor, multiple system atrophy, progressive supranuclear palsy, and/or vascular parkinsonism. In some embodiments, Parkinson's disease comprises immunologically mediated loss of dopaminergic cells. In some embodiments, Parkinson's disease does not comprise immunologically mediated loss of dopaminergic cells.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “subject,” as used herein, may be used interchangeably with the term “individual” and generally refers to an individual in need of a therapy. The subject can be a mammal, such as a human, dog, cat, horse, pig or rodent. The subject can be a patient, e.g., have or be suspected of having or at risk for having a disease or medical condition. For subjects having or suspected of having a medical condition directly or indirectly associated with bone, the medical condition may be of one or more types. The subject may have a disease or be suspected of having the disease. The subject may be asymptomatic. The subject may be of any gender, race, or age. The subject may be of a certain age, such as at least 30, 35, 40, 45, 50, 55, 60, 65, 70, 75, 80, 85, 90, 95, or 100 or more.

As used herein, the phrase “subject in need thereof” or “individual in need thereof” refers to a subject or individual, as described infra, that suffers or is at a risk of suffering (e.g, pre-disposed such as genetically pre-disposed, or subjected to environmental conditions that pre-dispose, etc.) from the diseases or conditions listed herein (e.g, Parkinson's Disease).

As used herein, the term “therapeutically effective amount” is synonymous with “effective amount”, “therapeutically effective dose”, and/or “effective dose” and refers to the amount of compound, cell, or other therapeutic that will elicit the biological, cosmetic or clinical response being sought by the practitioner in an individual in need thereof. This can be an amount of an agent sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. Effective amount can also mean the amount of a compound, material, or composition comprising a compound of the present disclosure that is effective for producing some desired effect, e.g., preventing binding of coronavirus to cell surface adhesion molecules. As one example, an effective amount is the amount sufficient to ameliorate and/or reverse Parkinson's disease in an individual. In one example, an effective amount of cells (e.g., dopaminergic cells or fibroblasts) is an amount of cells effective to regenerate or repair neural tissue in an individual having or at risk for developing Parkinson's disease. In another example, an effective amount of cells is an amount of cells capable of improving the behavior and/or neurological function of an individual having Parkinson's disease. An effective amount of cells may be between 10³ and 10¹¹ cells. In some cases, an effective amount of cells is about 10⁴ cells.

The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly. Further, one of skill in the art recognizes that an amount may be considered effective even if the medical condition is not totally eradicated but improved partially. For example, the medical condition may be halted or reduced or its onset delayed, a side effect from the medical condition may be partially reduced or completed eliminated, and so forth.

As used herein, the terms “treatment,” “treat,” or “treating” refers to intervention in an attempt to alter the natural course of the individual or cell being treated, and may be performed either for prophylaxis or during the course of pathology of a disease or condition such as for example Parkinson's Disease. Treatment may serve to accomplish one or more of various desired outcomes, including, for example, preventing occurrence or recurrence of disease, alleviation of symptoms, and diminishment of any direct or indirect pathological consequences of the disease, lowering the rate of disease progression, amelioration or palliation of the disease state, and remission or improved prognosis, and/or producing some desired effect, e.g., improving motor function.

### II. Fibroblasts and the Generation of Dopaminergic Cells from Fibroblasts

Aspects of the present disclosure relate to methods and compositions for generating one or more dopaminergic, or neuronal, cells from one or more fibroblasts. In some embodiments, fibroblasts are induced to differentiate into dopaminergic cells in vivo. Fibroblasts may be administered directly into the ventral mesencephalon, caudal diencephalic periaquaductal gray and/or hypothalamic regions of the brain where existing dopaminergic cells may induce differentiation of the fibroblasts into dopaminergic cells. In some embodiments, fibroblasts are induced to differentiate into dopaminergic cells in vitro. Dopaminergic cells may be generated by culturing fibroblasts under sufficient conditions to generate a dopaminergic cell. In some embodiments, the conditions comprise treatment of the fibroblasts with one or more agents capable of activating tyrosine hydroxylase expression in the fibroblasts.

In some embodiments, fibroblasts of the present disclosure are used as precursor cells that differentiate for example into dopaminergic cells following introduction into an individual. In some embodiments, fibroblasts are subjected to differentiation into a different cell type (e.g., a hematopoietic cell) prior to introduction into the individual.

In some embodiments, dopaminergic cells are generated from fibroblasts by culturing fibroblasts in growth media comprising all trans retinoic acid (RA) for a sufficient time. Fibroblasts may be cultured in a concentration of RA between 1 and 20 μM. In some embodiments, fibroblasts are cultured in at least 1, 2, 3, 4, 5, 6, 7, 8, 9, 10, 11, 12, 13, 14, 15, 16, 17, 18, 19, or 20 μM RA, or more. In some embodiments, fibroblasts are cultured in at most 20, 19, 18, 17, 16, 15, 14, 13, 12, 11, 10, 9, 8, 7, 6, 5, 4, 3, 2, or 1 μM RA, or less. In some embodiments, fibroblasts are cultured in about 1, about 2, about 3, about 4, about 5, about 6, about 7, about 8, about 9, about 10, about 11, about 12, about 13, about 14, about 15, about 16, about 17, about 18, about 19, or about 20 μM RA. In some embodiments, fibroblasts are cultured in about 10 μM RA. In some embodiments, fibroblasts are cultured in RA for between 1 and 6 weeks, between 2 and 6 weeks, between 3 and 6 weeks, between 4 and 6 weeks, or between 5 and 6 weeks. In some embodiments, fibroblasts are cultured in RA for about 1 week, about 2 weeks, about 3 weeks, about 4 weeks, about 5 weeks, about 6 weeks, about 7 weeks, about 8 weeks, or about 9 weeks. In some embodiments, fibroblasts are cultured in RA for between 5 weeks and 6 weeks.

In some embodiments, fibroblasts are further cultured with one or more growth factors and/or one or more neurotrophic factors. In some embodiments, fibroblasts are cultured with one or more of brain-derived neurotrophic factor (BDNF), glial cell line-derived neurotrophic factor (GDNF), nerve growth factor (NGF), and fibroblast growth factor (FGF).

In some embodiments, the dopaminergic cell expresses tyrosine hydroxylase, and tyrosine hydroxylase expression by the dopaminergic cell can be transient. In some embodiments, the dopaminergic cell expresses NeuN. In some embodiments, the dopaminergic cell does not express CD40. In some embodiments, the dopaminergic cell expresses Nestin.

Cells cultured with RA and/or one or more growth factors may be harvested (e.g., with trypsin) and re-plated at reduced density. Re-plated cells may be maintained in growth media for sufficient time (e.g., about 1 day, about 2 days, about 3 days, about 4 days, or about 5 days) and neuronal (e.g., dopaminergic) cells separated from accessory cells by gentle selective harvest, thereby obtaining an enriched neuron population. Following selective harvest, cells may be plated in media containing mitotic inhibitors for sufficient time (e.g., between 5 days and 10 days), after which purified neurons may be selectively harvested (e.g., with trypsin). Examples of mitotic inhibitors which may be used in enriching for neuronal cells include cytosine arabinoside (Ara-C) and 5-fluorodeoxyuridine (FUdR). Following purification, harvested cells may comprise at least 90% (e.g., >95%) neuronal cells (e.g., dopaminergic cells). Cells may be formulated in freezing media and cryopreserved.

Compositions of the present disclosure may be obtained from isolated fibroblast cells or a population thereof capable of proliferating and differentiating into ectoderm, mesoderm, or endoderm. In some embodiments, the fibroblasts possess the ability to differentiate to osteogenic, chondrogenic, and adipogenic lineage cells. In some embodiments, the enriched population of fibroblast cells are about 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 micrometers in size. In∥_{[NRF1]} some embodiments, fibroblasts of the present disclosure are adherent to plastic.

In some embodiments, the fibroblast cells are expanded and/or differentiated in culture using one or more cytokines, chemokines and/or growth factors prior to administration to an individual in need thereof. The agent capable of inducing fibroblast expansion can be selected from TPO, SCF, IL-1, IL-3, IL-7, flt-3L, G-CSF, GM-CSF, Epo, FGF-1, FGF-2, FGF-4, FGF-20, VEGF, activin-A, IGF, EGF, NGF, LIF, PDGF, and a member of the bone morphogenic protein family. The agent capable of inducing fibroblast differentiation can be selected from HGF, BDNF, VEGF, FGF1, FGF2, FGF4, and FGF20.

In some embodiments, the fibroblasts express CD73, CD90, and/or CD105. In some embodiments, the fibroblasts are CD14, CD34, CD45, and/or HLA-DR negative. In some embodiments, the fibroblast cells express proteins characteristic of normal fibroblasts including the fibroblast-specific marker, CD90 (Thy-1), a 35 kDa cell-surface glycoprotein, and the extracellular matrix protein, collagen. In some embodiments, an isolated fibroblast cell expresses at least one of Oct-4, Nanog, Sox-2, KLF4, c-Myc, Rex-1, GDF-3, LIF receptor, CD105, CD117, CD344, and/or Stella markers. In some embodiments, fibroblasts of the present disclosure express telomerase, Nanog, Sox2, β-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-α, HGF, c-MET, α-1-Antriptrypsin, Ceruloplasmin, AFP, PEPCK 1, BDNF, NT-4/5, TrkA, BMP2, BMP4, FGF2, FGF4, PDGF, PGF, TGFα, TGFβ, and/or VEGF. In some embodiments, an isolated fibroblast cell does not express at least one of MHC class I, MHC class II, CD45, CD13, CD49c, CD66b, CD73, CD105, and/or CD90 cell surface proteins. In some embodiments, the fibroblast cells do not produce one or more of CD31, CD34, CD45, CD117, CD141, HLA-DR, HLA-DP, HLA-DQ, or a combination thereof. In further embodiments, the fibroblast regenerative cell has enhanced expression of GDF-11 as compared to a control. In still further embodiments, the fibroblast cells express CD73, which is indicative of fibroblast cells having regenerative activity.

In some embodiments, the method optionally includes the step of depleting cells expressing stem cell surface markers or MHC proteins from the cell population, thereby isolating a population of stem cells. In some embodiments, the cells to be depleted express MHC class I, CD66b, glycophorin a, and/or glycophorin b. In some embodiments, fibroblast cells are isolated and expanded and possess one or more markers selected from a group consisting of CD10, CD13, CD44, CD73, CD90, CD141, PDGFr-α, HLA-A, HLA-B, HLA-C, or a combination thereof.

In some cases, fibroblast cells are obtained from a biopsy, and the donor providing the biopsy may be either the individual to be treated (autologous), or the donor may be different from the individual to be treated (allogeneic). In some embodiments, the fibroblast cells are xenogenic with respect to a recipient individual. In some embodiments wherein allogeneic fibroblast cells are utilized for an individual, the fibroblast cells may come from one or a plurality of donors. In some embodiments fibroblasts are used from young (less than 25 years old) donors. In some embodiments, steps are taken to protect allogeneic or xenogenic cells from immune-mediated rejection by the recipient. Steps include encapsulation, co-administration of an immune suppressive agent, transfection of said cells with immune suppressory agent, or a combination thereof. In some embodiments, tolerance to the cells is induced through immunological means. In some embodiments, fibroblasts are transfected with genes to allow for enhanced growth and to overcome the Hayflick limit. Subsequent to derivation by biopsy, the fibroblasts are expanded in culture using standard cell culture techniques.

In one embodiment, biopsies are from skin tissue (dermis and epidermis layers) from a subject's post-auricular area. In one embodiment, the starting material is composed of three 3-mm punch skin biopsies collected using standard aseptic practices, though the methods disclosed herein for preparing a skin biopsy tissue would apply equally to biopsies of other tissue types. The biopsies are collected by the treating physician, placed into a vial containing sterile phosphate buffered saline (PBS), and stored at 2-8° C. Upon initiation of the process, biopsies are inspected, and accepted biopsy tissues are washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Other commercially available collagenases may also be used, such as Serva Collagenase NB6 (Helidelburg, Germany).

After digestion, Iscove's Complete Growth Media (IMDM, GA, 10% Fetal Bovine Serum (FBS)) is added to neutralize the enzyme, and cells are pelleted by centrifugation and re-suspended in 5.0 mL IMDM. Alternatively, full enzymatic inactivation is achieved by adding IMDM without centrifugation. Additional IMDM is added prior to seeding of the cell suspension into a particular flask (such as a T-175 cell culture flask) for initiation of cell growth and expansion. Alternatively, a T-75, T-150, T-185, or T-225 flask can be used in place of the T-175 flask. Cells are incubated at 37.0±2° C. with 5.0±1.0% CO₂ and supplemented with fresh IMDM every three to five days by removing half of the IMDM and replacing it with the same volume with fresh media. Alternatively, full IMDM replacements can be performed.

In specific cases, cells are not cultured in the T-175 flask for more than 30 days prior to passaging. Confluence is monitored throughout the process to ensure adequate seeding densities upon culture splitting. When cell confluence is greater than or equal to 40% in the T-175 flask, the cells are passaged by removing the spent media, washing the cells, and treating the cells with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF), or a Two Layer Cell Stack (2 CS) can be used in place of the T-500 Flask.

Morphology may be evaluated at each passage and prior to harvest to monitor culture purity throughout the process by comparing the observed sample with visual standards for morphological examination of cell cultures. Typical fibroblast morphologies when growing in cultured monolayers include elongated, fusiform, or spindle-shaped cells with slender extensions or larger, flattened stellate cells with cytoplasmic leading edges. A mixture of these morphologies may also be observed. Fibroblasts in less confluent areas can be similarly shaped but randomly oriented. The presence of keratinocytes in cell cultures may also be evaluated. Keratinocytes are round and irregularly shaped and, at higher confluence, appear to be organized in a cobblestone formation. At lower confluence, keratinocytes are observable in small colonies.

Cells are incubated at 37±2° C. with 5.0±1.0% CO₂ and passaged every three to five days for cells in T-500 flasks and every five to seven days for cells in ten layer cell stacks (10 CS). Cells should not be cultured for more than 10 days prior to passaging. When cell confluence in a T-500 flask is ≥95%, cells are passaged to a 10 Layer Cell Stack (10 CS) culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flask into the solution. Cells are then transferred to the 10 CS. Additional IMDM is added to neutralize the trypsin, and the cells from the T-500 flask are pipetted into a 2 L bottle containing fresh IMDM. The contents of the 2 L bottle may be transferred into the 10 CS and seeded across all layers of the 10 CS. Cells are then incubated at 37.0±2° C. with 5.0±1.0% CO₂ and supplemented with fresh IMDM every five to seven days. In some cases, cells should not be cultured in the 10 CS for more than 20 days prior to passaging. In one embodiment, the passaged fibroblasts are rendered substantially free of immunogenic proteins present in the culture medium by incubating the expanded fibroblasts for a period of time in protein-free medium. When cell confluence in the 10 CS is ≥95%, cells are harvested. Harvesting is performed by removing the spent media, washing the cells, treating with Trypsin-EDTA to release adherent cells into the solution, and adding additional IMDM to neutralize the trypsin. Cells are collected by centrifugation and resuspended, and quality control testing is performed to determine total viable cell count and cell viability as well as sterility and the presence of endotoxins.

The fibroblasts may also be obtained from a source selected from the group consisting of: dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and a combination thereof. In some embodiments, fibroblasts are fibroblasts isolated from placenta, cord blood, peripheral blood, omentum, hair follicle, skin, bone marrow, adipose tissue, or Wharton's Jelly.

In some embodiments, the fibroblasts are fibroblasts isolated from peripheral blood of a subject who has been exposed to conditions sufficient to stimulate fibroblasts from the subject to enter the peripheral blood. In another embodiment, fibroblast cells are mobilized by use of a mobilizing agent or therapy for treatment of ovarian failure. In some embodiments, the conditions and/or agents sufficient to stimulate fibroblasts from the subject to enter the peripheral blood comprise administration of VLA 5 antibodies, G-CSF, M-CSF, GM-CSF, TNF-α, 5-FU, IL-1, IL-3, kit-L, VEGF, Flt-3 ligand, PDGF, EGF, FGF-1, FGF-2, FGF-5, TPO, IL-11, IGF-1, MGDF, NGF, HMG CoA reductase inhibitors, small molecule antagonists of SDF-1, or a combination thereof. In some embodiments, the mobilization therapy is selected from a group comprising exercise, hyperbaric oxygen, autohemotherapy by ex vivo ozonation of peripheral blood, induction of SDF-1 secretion in an anatomical area outside of the bone marrow, or a combination thereof. In some embodiments, the committed fibroblasts can express the marker CD133 or CD34 and are mobilized.

Any of the fibroblast cell populations disclosed herein may be used as a source of conditioned media. The cells may be cultured alone, or may by cultured in the presence of other cells in order to further upregulate production of growth factors in the conditioned media. In some embodiments, fibroblasts of the present disclosure are cultured with an inhibitor of mRNA degradation. In some embodiments, fibroblasts are cultured under conditions suitable to support differentiation and/or reprogramming of the fibroblasts. In some embodiments, such conditions comprise temperature conditions of between 30° C. and 38° C., between 31° C. and 37° C., or between 32° C. and 36° C. In some embodiments, such conditions comprise glucose at or below 4.6 g/l, 4.5 g/l, 4 g/l, 3 g/l, 2 g/l or 1 g/l. In some embodiments, such conditions comprise glucose of about 1 g/l.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number, or the “doubling time.” Fibroblast cells used in the disclosed methods can undergo at least 25, 30, 35, or 40 doublings prior to reaching a senescent state. Methods for deriving cells capable of doubling to reach 10¹⁴ cells or more are provided. Preferred are those methods which derive cells that can double sufficiently to produce at least about 10¹⁴, 10¹⁵, 10¹⁶, or 10¹⁷ or more cells when seeded at from about 10³ to about 10⁶ cells/cm² in culture. Preferably these cell numbers are produced within 80, 70, or 60 days or less.

When referring to cultured cells, including fibroblast cells and vertebrae cells, the term senescence (also “replicative senescence” or “cellular senescence”) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are resistant to programmed cell death (apoptosis) and can be maintained in their nondividing state for as long as three years. These cells are alive and metabolically active, but they do not divide.

Fibroblast cells used in the disclosed methods can undergo at least 25, 30, 35, or 40 doublings prior to reaching a senescent state. Methods for deriving cells capable of doubling to reach 10¹⁴ cells or more are provided. In some embodiments, methods are used to derive cells that can double sufficiently to produce at least about 10¹⁴, 10¹⁵, 10¹⁶, or 10¹⁷ or more cells when seeded at from about 10³ to about 10⁶ cells/cm² in culture within 80, 70, or 60 days or less. In some embodiments, fibroblasts are transfected with one or more genes to allow for enhanced growth and overcoming of the Hayflick limit.

As disclosed herein, fibroblasts may secrete one or more factors prior to or following introduction into an individual. Such factors include, but are not limited to, growth factors, trophic factors and cytokines. In some instances, the secreted factors can have a therapeutic effect in the individual. In some embodiments, a secreted factor activates the same cell. In some embodiments, the secreted factor activates neighboring and/or distal endogenous cells. In some embodiments, the secreted factor stimulated cell proliferation and/or cell differentiation. In some embodiments, fibroblasts secrete a cytokine or growth factor selected from human growth factor, fibroblast growth factor, nerve growth factor, insulin-like growth factors, hematopoietic stem cell growth factors, a member of the fibroblast growth factor family, a member of the platelet-derived growth factor family, a vascular or endothelial cell growth factor, and a member of the TGFβ family.

In some embodiments, fibroblasts are manipulated or stimulated to produce one or more factors. In some embodiments, fibroblasts are manipulated or stimulated to produce leukemia inhibitory factor (LIF), brain-derived neurotrophic factor (BDNF), epidermal growth factor receptor (EGF), basic fibroblast growth factor (bFGF), FGF-6, glial-derived neurotrophic factor (GDNF), granulocyte colony-stimulating factor (GCSF), hepatocyte growth factor (HGF), IFN-γ, insulin-like growth factor binding protein (IGFBP-2), IGFBP-6, IL-1ra, IL-6, IL-8, monocyte chemotactic protein (MCP-1), mononuclear phagocyte colony-stimulating factor (M-CSF), neurotrophic factors (NT3), tissue inhibitor of metalloproteinases (TIMP-1), TIMP-2, tumor necrosis factor (TNF-β), vascular endothelial growth factor (VEGF), VEGF-D, urokinase plasminogen activator receptor (uPAR), bone morphogenetic protein 4 (BMP4), IL1-a, IL-3, leptin, stem cell factor (SCF), stromal cell-derived factor-1 (SDF-1), platelet derived growth factor-BB (PDGFBB), transforming growth factors beta (TGFβ-1) and/or TGFβ-3. Factors from manipulated or stimulated fibroblasts may be present in conditioned media and collected for therapeutic use.

### III. Dopaminergic Cells for Parkinson's Disease Treatment

Certain aspects of the present disclosure relate to the use of dopaminergic cells, such as dopaminergic cells generated from fibroblasts, for treatment or prevention of Parkinson's disease in an individual. Methods for generation of dopaminergic cells from fibroblasts are described elsewhere herein. In some embodiments, the disclosed methods comprise providing an effective amount of dopaminergic cells to an individual sufficient to treat Parkinson's disease. Dopaminergic cells may produce dopamine in the individual, thereby reducing the severity of Parkinson's disease in the individual.

Cell therapy for patients with Parkinson's disease has been previously used, and methods of administering cell therapy, as well as methods of specifically selecting patients for therapy, including a study in which patients underwent stereotaxic implantation of human fetal ventral mesencephalic tissue in one caudate nucleus. Individuals treated with dopaminergic cells of the present disclosure may be selected based on certain inclusion criteria. For example, criteria for inclusion may be age of 35 years or more, three or four cardinal signs of Parkinson's disease (resting tremor, rigidity, bradykinesia, or postural instability), and Hoehn-Yahr stage 4 parkinsonian disability at some point daily. In some embodiments, patients selected for treatment using the disclosed methods may be responsive to levodopa but have had had treatment failure or unacceptable side effects. Other patient inclusion/exclusion criteria known to those of skill in the art may be utilized. Other tissues and cells may be transplanted into Parkinson's disease patients. For example, transplanted tissues and cells can include fetal derived dopaminergic cells, fetal mesencephalic/nigra substantia tissue, xenogeneic fetal mesencephalic tissue, thoracic sympathetic trunk neurons, bone marrow mesenchymal stem cells, neural progenitor cells, and retinal pigment epithelial cells.

In some embodiments, dopaminergic cells generated from fibroblasts are administered to an individual in a manner sufficient to achieve homogenous reinnervation of the striatum. Therefore, in some embodiments, administration of dopaminergic cells may comprise placement of needle tracts in the putamen of the individual at various intervals. In some embodiments, needle tracts are placed at approximately 4 mm intervals. Needle size may be important for graft survival. In some embodiments, a cannula (e.g., a 1.5 mm cannula) is extended from its support in a stereotactic apparatus to the surface of the brain of an individual, wherein an inner stylet with an outer diameter of between 0.46 and 0.64 mm penetrates the putamen of the individual. Such a needle may be used to deliver dopaminergic cells generated from fibroblasts to the individual. In some embodiments, before administration of cells, the caudate or putamen of the individual is visualized, for example, by CT scanning. Typically, the long axis of putamen is at least 30 mm long, a length suitable for six or seven needle passes on each side. Implantation may be carried out through elliptical craniectomy. In some embodiments, an individual may be awake and sedated under local anesthesia. In other embodiments, an individual may be treated under general anesthesia. In some embodiments, a rotating template is used to create a row of up to nine needle tracts, with a single set of coordinates for the center tract in the row. Deposits of cells may be made along 10 mm tracts in the putamen or caudate (or both) as the needle is slowly withdrawn.

In some embodiments, in addition to dopaminergic cells, cyclosporine is administered to an individual. In addition to potential antirejection effects of cyclosporine (CsA), which in some embodiments may not be required for treatment of Parkinson's disease, cyclosporine may be added to enhance neurological function of transplanted cells. Various examples of neurological uses of cyclosporine are provided, any of which may be used to facilitate administration of cyclosporine together with dopaminergic cells of the present disclosure. Without wishing to be bound by theory, CsA may work through blocking mitochondrial permeability transition pore (MPTP) opening, which has been demonstrated in animal models of CNS disorders, including stroke, traumatic brain injury, and Parkinson's disease (PD). CsA may act through binding with cyclophilin-calcineurin (CN) complex to suppress cytokine gene expression and block of T lymphocyte action. CsA is the treatment of choice for immunosuppression in organ and neural transplantation. The inhibition of CN can result in the inhibition of nitric oxide synthase activation, free radical formation, or the mitochondria-induced cell death.

### IV. Fibroblasts for Parkinson's Disease Treatment

Aspects of the disclosure relate to use of fibroblasts for treatment or prevention of Parkinson's disease. In some embodiments, the disclosed methods comprise treating or preventing Parkinson's disease by providing an effective amount of fibroblasts. Fibroblasts may be dopaminergic fibroblasts. Fibroblasts may be administered to an individual to decrease immunological reactions associated with Parkinson's disease. The fibroblasts can be exposed to inflammatory conditions, and the fibroblasts can reduce inflammation in the individual.

In some embodiments, the individual has been diagnosed with Parkinson's disease. In some embodiments, the individual is at risk for developing Parkinson's disease. The individual at risk of developing Parkinson's disease includes male individuals, individuals age 60 and older, individuals with a genetic predisposition, individuals who have been exposed to environmental toxins, and/or individuals with a history of head trauma. In some embodiments, the Parkinson's disease comprises immunologically mediated loss of endogenous dopaminergic cells in the individual. In some embodiments, the Parkinson's disease comprises non-immunologically mediated loss of endogenous dopaminergic cells in the individual. In some embodiments, the Parkinson's disease comprises corticobasal degeneration, dementia with Lewy bodies, drug-induced parkinsonism, essential tremor, multiple system atrophy, progressive supranuclear palsy, vascular parkinsonism, or a combination thereof.

Studies have shown that Parkinson's disease possesses an immunological component in which antibodies and T cells are demonstrated to possess autoreactive properties. Supporting an immunological basis of PD is a study in which a hemiparkinsonian syndrome was induced in rats by unilateral injections of 6-hydroxydopamine into the pars compacta of the substantia nigra. Phytohemagglutinin-stimulated rat peritoneal cells, predominantly T cells and macrophages, were stereotactically implanted in the lesioned caudate-putamen, and amphetamine-induced turning was used to assess recovery. Animals receiving implants of activated peritoneal cells showed a 47% decrease in amphetamine-induced turning 8 weeks after implantation, which was not seen in control or sham-operated animals. Immunocytochemistry revealed increased tyrosine hydroxylase reactive fibers in the leukocyte-implanted striatum. The authors concluded that implantation of activated leukocytes promotes functional recovery in hemiparkinsonian rats.

Cell therapy for patients with Parkinson's disease has been previously used, and methods of administering cell therapy, as well as methods of specifically selecting patients for therapy, may be drawn from published methods, including a study in which patients underwent stereotaxic implantation of human fetal ventral mesencephalic tissue in one caudate nucleus. Individuals treated with fibroblasts of the present disclosure may be selected based on certain inclusion criteria. For example, criteria for inclusion may be age of 35 years or more, three or four cardinal signs of Parkinson's disease (resting tremor, rigidity, bradykinesia, or postural instability), and Hoehn-Yahr stage 4 parkinsonian disability at some point daily. In some embodiments, patients selected for treatment using the disclosed methods may be responsive to levodopa but have had had treatment failure or unacceptable side effects. Other patient inclusion/exclusion criteria known to those of skill in the art may be utilized. Other tissues and cells may be transplanted into Parkinson's disease patients. For example, transplanted tissues and cells can include fetal derived dopaminergic cells, fetal mesencephalic/nigra substantia tissue, xenogeneic fetal mesencephalic tissue, thoracic sympathetic trunk neurons, bone marrow mesenchymal stem cells, neural progenitor cells, and retinal pigment epithelial cells.

In some embodiments, fibroblasts are administered to an individual in a manner sufficient to achieve homogenous reinnervation of the striatum. Therefore, in some embodiments, administration of fibroblasts may comprise placement of needle tracts in the putamen of the individual at various intervals. In some embodiments, needle tracts are placed at approximately 4 mm intervals. Needle size may be important for graft survival. In some embodiments, a cannula (e.g., a 1.5 mm cannula) is extended from its support in a stereotactic apparatus to the surface of the brain of an individual, wherein an inner stylet with an outer diameter of between 0.46 and 0.64 mm penetrates the putamen of the individual. Such a needle may be used to deliver fibroblasts to the individual. In some embodiments, before administration of cells, the caudate or putamen of the individual is visualized, for example, by CT scanning. Typically, the long axis of putamen is at least 30 mm long, a length suitable for six or seven needle passes on each side. Implantation may be carried out through elliptical craniectomy. In some embodiments, an individual may be awake and sedated under local anesthesia. In other embodiments, an individual may be treated under general anesthesia. In some embodiments, a rotating template is used to create a row of up to nine needle tracts, with a single set of coordinates for the center tract in the row. Deposits of cells may be made along 10 mm tracts in the putamen or caudate (or both) as the needle is slowly withdrawn.

In some embodiments, in addition to fibroblasts, cyclosporine is administered to an individual. In addition to potential antirejection effects of cyclosporine (CsA), which in some embodiments may not be required for treatment of Parkinson's disease, cyclosporine may be added to enhance neurological function of transplanted cells. Various examples of neurological uses of cyclosporine are provided, any of which may be used to facilitate administration of cyclosporine together with dopaminergic cells of the present disclosure [2-22]. Without wishing to be bound by theory, CsA may work through blocking mitochondrial permeability transition pore (MPTP) opening, which has been demonstrated in animal models of CNS disorders, including stroke, traumatic brain injury, and Parkinson's disease (PD). CsA may act through binding with cyclophilin-calcineurin (CN) complex to suppress cytokine gene expression and block of T lymphocyte action. CsA is the treatment of choice for immunosuppression in organ and neural transplantation. The inhibition of CN can result in the inhibition of nitric oxide synthase activation, free radical formation or the mitochondria-induced cell death.

### V. Administration of Therapeutic Compositions

The therapy provided herein may comprise administration of therapeutic agents (e.g., fibroblasts, exosomes from fibroblasts, etc.) alone or in combination. Therapies may be administered in any suitable manner known in the art. For example, a first and second treatment may be administered sequentially (at different times) or concurrently (at the same time). In some embodiments, the first and second treatments are administered in a separate composition. In some embodiments, the first and second treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents (e.g., fibroblasts) of the disclosure may be administered by the same route of administration or by different routes of administration. In some embodiments, the therapy is administered intravenously, intramuscularly, subcutaneously, topically, orally, sublingually, transdermally, intraperitoneally, intraorbitally, by implantation, by inhalation, intrathecally, intraventricularly, and/or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

In some embodiments, between about 10⁵ and about 10¹³ cells per 100 kg are administered to a human per infusion. In some embodiments, between about 1.5×10⁶ and about 1.5×10¹² cells are infused per 100 kg. In some embodiments, between about 1×10⁹ and about 5×10¹¹ cells are infused per 100 kg. In some embodiments, between about 4×10⁹ and about 2×10¹¹ cells are infused per 100 kg. In some embodiments, between about 5×10⁸ cells and about 1×10¹ cells are infused per 100 kg. In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In some embodiments, 5 administrations are provided over the course of 5 consecutive days. In some embodiments, a single administration of between about 10⁵ and about 10¹³ cells per 100 kg is provided. In some embodiments, a single administration of between about 1.5×10⁸ and about 1.5×10¹² cells per 100 kg is provided. In some embodiments, a single administration of between about 1×10⁹ and about 5×10¹¹ cells per 100 kg is provided. In some embodiments, a single administration of about 5×10¹⁰ cells per 100 kg is provided. In some embodiments, a single administration of 1×10¹⁰ cells per 100 kg is provided. In some embodiments, multiple administrations of between about 10⁵ and about 10¹³ cells per 100 kg are provided. In some embodiments, multiple administrations of between about 1.5×10⁸ and about 1.5×10¹² cells per 100 kg are provided. In some embodiments, multiple administrations of between about 1×10⁹ and about 5×10¹¹ cells per 100 kg are provided over the course of 3-7 consecutive days. In some embodiments, multiple administrations of about 4×10⁹ cells per 100 kg are provided over the course of 3-7 consecutive days. In some embodiments, multiple administrations of about 2×10¹¹ cells per 100 kg are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

The quantity to be administered, both according to number of treatments and unit dose, depends on the treatment effect desired. An effective dose is understood to refer to an amount necessary to achieve a particular effect. In some embodiments, it is contemplated that doses between about 10⁵ and about 10¹³ cells per 100 kg are administered to affect the protective capability of the fibroblasts. In the practice in certain embodiments, it is contemplated that a cell number equivalent to doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain embodiments, the effective dose of a pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

### VI. Kits of the Disclosure

Any of the cellular and/or non-cellular compositions described herein or similar thereto may be comprised in a kit. In a non-limiting example, one or more reagents for use in methods for preparing fibroblasts or derivatives thereof may be comprised in a kit. Such reagents may include cells, vectors, one or more growth factors, vector(s), one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate particular embodiments of the disclosure. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Superior Proliferation of Fibroblasts Compared to Bone Marrow and Adipose-Derived Mesenchymal Stem Cells

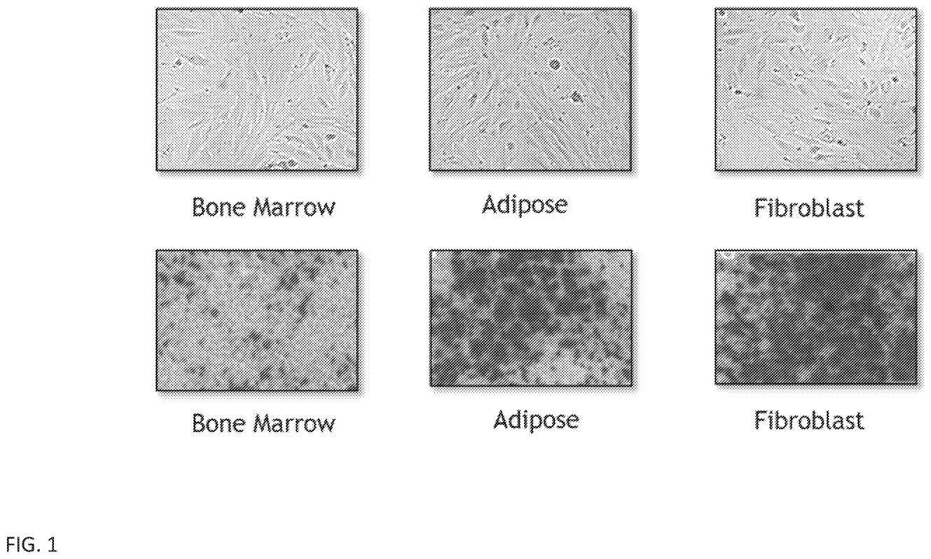

Dermal fibroblasts, bone marrow mesenchymal stem cells (BM-MSCs), and adipose derived mesenchymal stem cells (A-MSCs) where cultured for 7 days in DMEM with 10% fetal calf serum in the presence of 0.03 IU oxytocin per ml. Proliferation was assessed by MTT staining. FIG. 1 shows the results of the proliferation analysis. Significantly greater proliferation was observed for the fibroblast cells compared to the BM-MSC and A-MSC under these conditions. The fibroblast cells doubled once every 18-22 hours, while the BM-MSC doubled every 28-36 hours, and the A-MSC doubled every 25-29 hours.

### Example 2

### Differentiation of Fibroblasts into Cells Having Neuron-Like Morphology and Phenotype

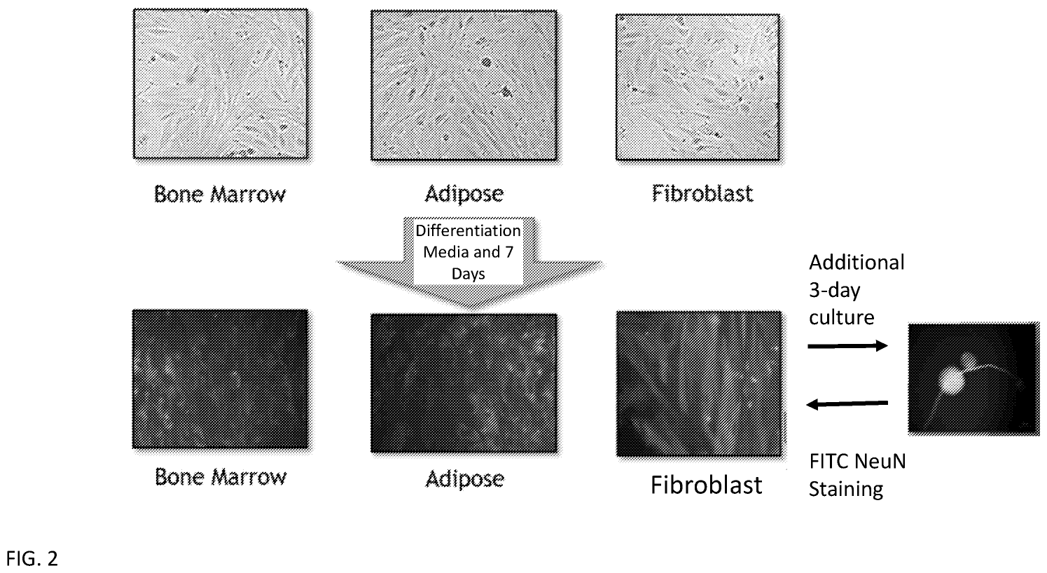

Fibroblasts, BM-MSCs and A-MSCs were plated in 6 well polylysine coated plates at a concentration of 50,000 cells per ml. Cells where incubated in induction media containing N2 media supplemented with 0.5 μM all-trans retinoic acid (RA) and 1 ng/ml brain-derived neurotrophic factor (BDNF). Cells where cultured for 7 days in a fully humidified atmosphere with addition of 0.03 IU oxytocin per ml. As shown in FIG. 2, increased NeuN staining as well as neuron-like morphology was observed in cultures from fibroblasts. Furthermore, an additional 3 day culture resulted in further enhancement of neuronal morphology in the fibroblast derived cells but not in the BM-MSCs or A-MSCs.

### Example 3

### Production of Tyrosine Hydroxylase by Fibroblasts

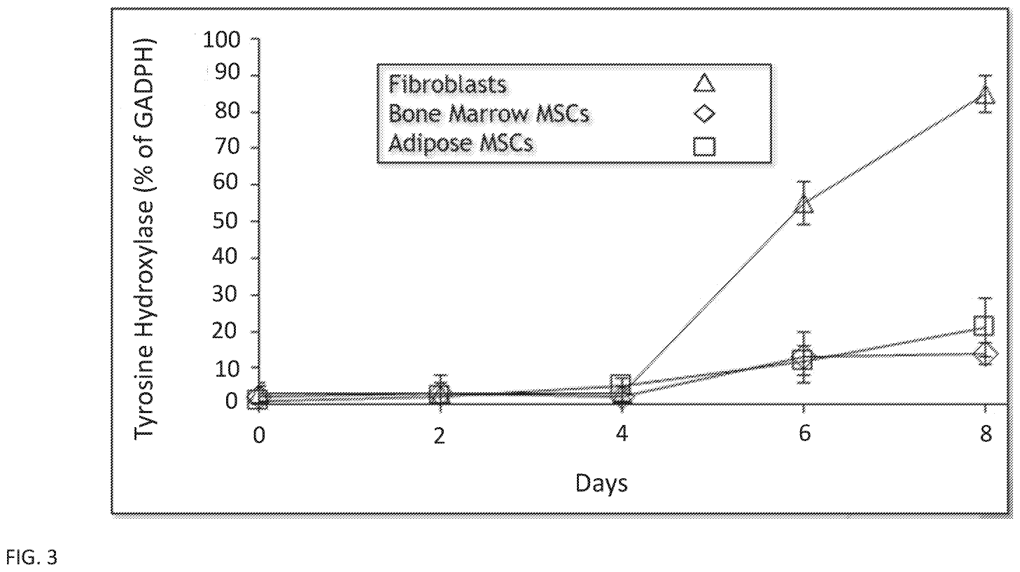

Cells were grown as described in Example 1 and 2, and induced to differentiate as described in Example 2, except that cells were cultured for 8 days. Tyrosine hydroxylase was assessed by quantitative real-time polymerase chain reaction (RT-PCR) and expressed as percentage of housekeeping gene GAPDH. As shown in FIG. 3, significant induction of this dopamine-producing enzyme was observed in the fibroblast-derived neuronal cells compared with the BM-MSCs and A-MSCs.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method for treating or preventing Parkinson's disease in an individual comprising administering an effective amount of a composition comprising fibroblast cells to an individual in need thereof.

2. The method of claim 1, wherein the fibroblast cells are dopaminergic fibroblasts cells.

3. The method of claim 1 or 2, wherein the fibroblast cells are cultured under conditions sufficient to differentiate the fibroblasts into neuronal cells.

4. The method of claim 3, wherein the conditions comprise treatment of the fibroblasts with one or more agents capable of activating tyrosine hydroxylase expression in the fibroblasts.

5. The method of claim 3 or 4, wherein the conditions comprise culturing the fibroblasts with all-trans retinoic acid (RA).

6. The method of any one of claims 2-5, wherein the conditions comprise culturing the fibroblasts with one or more growth factors.

7. The method of claim 6, wherein the growth factor is one or more neurotrophic factors.

8. The method of claim 7, wherein the one or more neurotrophic factors comprise brain-derived neurotrophic factor (BDNF), glial cell line-derived neurotrophic factor (GDNF), nerve growth factor (NGF), a fibroblast growth factor (FGF), or a combination thereof.

9. The method of any one of claims 1-8, wherein the Parkinson's disease comprises immunologically mediated loss of endogenous dopaminergic cells in the individual.

10. The method of any one of claims 1-9, wherein the Parkinson's disease comprises non-immunologically mediated loss of endogenous dopaminergic cells in the individual.

11. The method of any one of claims 1-10, wherein the dopaminergic cells express tyrosine hydroxylase.

12. The method of claim 11, wherein the dopaminergic cells express the tyrosine hydroxylase transiently.

13. The method of any one of claims 1-12, wherein the dopaminergic cells express NeuN.

14. The method of any one of claims 1-13, wherein the dopaminergic cells do not express CD40.

15. The method of any one of claims 1-14, wherein the dopaminergic cells express Nestin.

16. The method of any one of claims 1-15, wherein the dopaminergic cells are provided to the individual intracranially.

17. The method of claim 16, wherein the dopaminergic cells are provided to the putamen of the individual.

18. The method of any one of claims 1-17, wherein the individual has been diagnosed with Parkinson's disease.

19. The method of any one of claims 1-17, wherein the individual is at risk for developing Parkinson's disease.

20. The method of claim 19, wherein the individual at risk of developing Parkinson's disease includes male individuals, individuals age 60 and older, individuals with a genetic predisposition, individuals who have been exposed to environmental toxins, and/or individuals with a history of head trauma.

21. The method of any one of claims 1-20, wherein the Parkinson's disease comprises corticobasal degeneration, dementia with Lewy bodies, drug-induced parkinsonism, essential tremor, multiple system atrophy, progressive supranuclear palsy, vascular parkinsonism, or a combination thereof.

21. The method of any one of claims 1-21, wherein the fibroblasts are exposed to inflammatory conditions, and wherein the fibroblasts reduce inflammation in the individual.

22. A method for generating dopaminergic cells from fibroblasts comprising subjecting fibroblasts to conditions sufficient to generate dopaminergic cells from the fibroblasts.

23. The method of claim 22, wherein the conditions comprise conditions sufficient to differentiate the fibroblasts into neuronal cells.

24. The method of claim 22 or 23, wherein the conditions comprise treatment of the fibroblasts with one or more agents capable of activating tyrosine hydroxylase expression in the fibroblasts.

25. The method of any one of claims 22-24, wherein the conditions comprise culturing the fibroblasts with all-trans retinoic acid (RA).

26. The method of claim 25, wherein the conditions further comprise culturing the fibroblasts with one or more growth factors.

27. The method of claim 26, wherein the growth factor is one or more neurotrophic factors.

28. The method of claim 27, wherein the one or more neurotrophic factors comprise brain-derived neurotrophic factor (BDNF), glial cell line-derived neurotrophic factor (GDNF), nerve growth factor (NGF), a fibroblast growth factor (FGF), or a combination thereof.

29. The method of any one of claims 22-28, wherein the dopaminergic cells express tyrosine hydroxylase.

30. The method of claim 29, wherein the dopaminergic cells express the tyrosine hydroxylase transiently.

31. The method of any one of claims 22-30, wherein the dopaminergic cells express NeuN.

32. The method of any one of claims 22-31, wherein the dopaminergic cells do not express CD40.

33. The method of any one of claims 22-32, wherein the dopaminergic cells express Nestin.

34. The method of any one of claims 1-33, wherein the fibroblasts are fibroblasts isolated from placenta, cord blood, peripheral blood, omentum, hair follicle, skin, bone marrow, adipose tissue, or Wharton's Jelly.

35. The method of any one of claims 1-34, wherein the fibroblasts are fibroblasts isolated from peripheral blood of a subject who has been exposed to conditions sufficient to stimulate fibroblasts from the subject to enter the peripheral blood.

36. The method of claim 35, wherein the conditions sufficient to stimulate fibroblasts from the subject to enter the peripheral blood comprise administration of VLA-5 antibodies, G-CSF, M-CSF, GM-CSF, FLT-3L, TNF-α, EGF, FGF-1, FGF-2, FGF-5, VEGF, or a combination thereof.

37. A dopaminergic cell, wherein the dopaminergic cell is derived from a fibroblast that was cultured with all-trans retinoic acid (RA) and one or more neurotrophic factors.

38. The dopaminergic cell of claim 37, wherein the dopaminergic cell expresses tyrosine hydroxylase.

39. The dopaminergic cell of claim 38, wherein the dopaminergic cell expresses the tyrosine hydroxylase transiently.

40. The dopaminergic cell of any one of claims 37-39, wherein the dopaminergic cell expresses NeuN.

41. The dopaminergic cell of any one of claims 37-40, wherein the dopaminergic cell does not express CD40.

42. The dopaminergic cell of any one of claims 37-41, wherein the dopaminergic cell expresses Nestin.

43. The dopaminergic cell of any one of claims 37-42, wherein the one or more neurotrophic factors comprise brain-derived neurotrophic factor (BDNF), glial cell line-derived neurotrophic factor (GDNF), nerve growth factor (NGF), a fibroblast growth factor (FGF), or a combination thereof.

44. The dopaminergic cell of any one of claims 37-43, wherein the fibroblast was isolated from placenta, cord blood, peripheral blood, omentum, hair follicle, skin, bone marrow, adipose tissue, or Wharton's Jelly.

45. The dopaminergic cell of any one of claims 37-44, wherein the fibroblast was isolated from peripheral blood of a subject who has been exposed to conditions sufficient to stimulate fibroblasts from the subject to enter the peripheral blood.

46. The dopaminergic cell of claim 45, wherein the conditions sufficient to stimulate fibroblasts from the subject to enter the peripheral blood comprise administration of VLA-5 antibodies, G-CSF, M-CSF, GM-CSF, FLT-3L, TNF-α, EGF, FGF-1, FGF-2, FGF-5, VEGF, or a combination thereof.
